(19) United States
(12) Patent Application Publication
(10) Pub. No.: US 2012/0283298 A1
(43) Pub. Date: Nov. 8, 2012

(54) METHODS FOR TREATING MEMORY IMPAIRMENT

(76) Inventors: Albert Michael Crider, Edwardsville, IL (US); Kenneth A. Witt, Edwardsville, IL (US); Karin E. Sandoval, Edwardsville, IL (US)

(21) Appl. No.: 13/502,364

(22) PCT Filed: Oct. 14, 2010
(86) PCT No.: PCT/US10/52688

§ 371 Date: Jul. 27, 2012

Related U.S. Application Data
(60) Provisional application No. 61/252,506, filed on Oct. 16, 2009.

(51) Int. Cl. A61K 31/4178; C07D 403/12; A61P 25/00; A61K 31/4439

(52) U.S. Cl. 514/341; 514/397; 548/312.1

(57) ABSTRACT

Methods for administering a therapeutically effective amount of a somatostatin receptor-4 (SSTR4) agonist in order to treat learning and memory impairment for Alzheimer's disease is disclosed.

### FIELD OF THE INVENTION

The present disclosure relates to medical treatment methods and more particularly to methods for treating the learning and memory impairment of Alzheimer's disease.

### BACKGROUND OF THE INVENTION

Alzheimer's disease (AD) is a neurodegenerative disorder affecting more than 20 million individuals worldwide. AD is associated with increases in brain beta amyloid (All) levels and a progressive loss in memory and other cognitive abilities. To date, the only available therapeutic agents for treating AD provide only short-term clinical benefits and do not prevent actual disease progression.

Somatostatin (somatotropin release-inhibiting factor, SRIF) is a peptide widely expressed throughout the central nervous system that is a primary mediator of All degradation through stimulation of the enzyme neprilysin (NEP). NEP is a zinc-dependent metalloprotease enzyme that degrades a number of small secreted peptides and has shown to regulate the steady-state levels of All levels in the brain with an affinity (Km) for All1-42 of 7 μM. Somatostatin levels in the brain have been shown to drop as low as 10-20% in association with aging and AD progression. It has been hypothesized that somatostatin levels decrease with increased age, resulting in a corresponding decrease in NEP activity and/or expression (Hama and Saido, 2005). Subsequently, the decrease in NEP may cause steady-state All levels in the brain to increase. The All1-42 form has been consistently identified with amyloid plaque formation associated with neuronal death and development of AD (Selkoe, 2008). NEP expression has also been shown to decrease in the brain during aging and in the early stages of AD, while elevated NEP activity has been shown to reduce the accumulation of both soluble and insoluble All in amyloid-precursor protein (APP) transgenic mice. Thus, drugs designed to enhance specific somatostatin receptor subtype activity in the brain may provide a selective and effective means for AD treatment by enhancing the expression and/or activity of neprilysin.

To date, five somatostatin receptor (SSTR) subtypes have been identified, all of which are G-protein coupled receptors. These receptor subtypes are distributed in distinct patterns within the body. Among the five somatostatin receptor subtypes, SSTR4 has been shown to be heavily expressed in the neocortex and hippocampus (see, e.g., Moller et al. 2003), areas critical in learning and memory. Additionally, these areas are significantly affected by AR accumulation and associated with reduced NEP levels in AD patients.

### SUMMARY OF THE INVENTION

In one aspect, the present disclosure provides a method for treating memory impairment from Alzheimer's disease in a subject in need thereof, comprising administering to the subject a therapeutically effective amount of a somatostatin receptor-4 (SSTR4) agonist.

In another aspect, the present disclosure provides a method of improving memory function in a subject suffering from Alzheimer's disease, the method comprising: a. determining a first level of memory function in the subject; b. administering to the subject a therapeutically effective amount of a somatostatin receptor-4 (SSTR4) agonist; and c. determining a second level of memory function following administration of the SSTR4 agonist; d. comparing the second level of memory function in the subject to the first level of memory function in the subject such that a detectable improvement in memory function indicates that the subject is responding positively to the administration of the SSTR4 agonist.

In another aspect, the present disclosure provides a method to improve memory function in a subject suffering from Alzheimer's disease, the method comprising reducing Aβ42 levels in brain tissue of the subject by administering to the subject a therapeutically effective amount of a somatostatin receptor-4 (SSTR4) agonist.

In any of the methods, the SSTR4 agonist can be a compound of formula I:

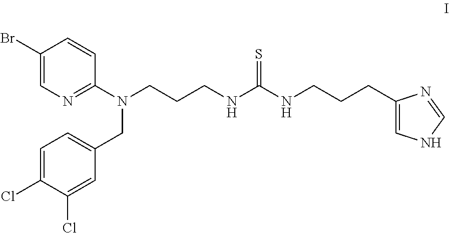

Alternatively, the SSTR4 agonist can be a compound of formula II:

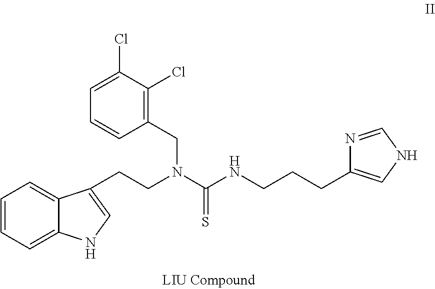

Alternatively, the SSTR4 agonist can be a compound of formula III (3-(3-1H-Imidazol-4-yl)propyl-1-(2-(1H-indol-3-yl)ethyl-1-(3,4-dichlorobenzyl)urea):

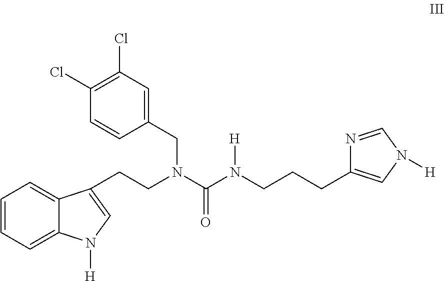

In any of the methods, the therapeutically effective amount can be from 0.01 mg/kg to about 10 mg/kg of body weight per day. The somatostatin receptor-4 (SSTR4) agonist can be administered to the subject through a peripheral administration route, which can be selected from the group consisting of intraperitoneal, intravenous, intramuscular and intraarterial. The somatostatin receptor-4 (SSTR4) agonist can be administered in an intravenous dosage form further comprising a pharmaceutically acceptable carrier, or in an intraperitoneal dosage form further comprising a pharmaceutically acceptable carrier.

In another aspect, the present disclosure provides a compound of formula III (“3-(3-1H-Imidazol-4-yl)propyl-1-(2-(1H-indol-3-yl)ethyl-1-(3,4-dichlorobenzyl)urea”):

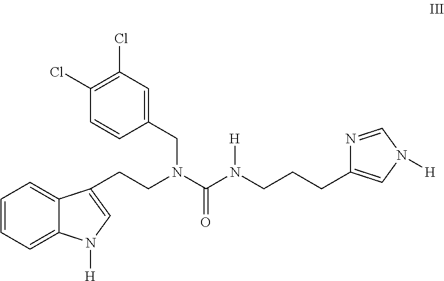

The compound of Formula III can be combined with a pharmaceutically acceptable carrier, excipient or diluent to form a pharmaceutical composition.

### BRIEF DESCRIPTION OF THE DRAWINGS

FIGS. 1A and 1B shows bar graphs of results obtained when acquisition (learning) of SAMP8 mice was evaluated via T-maze task 24 h following i.c.v. injection of (A) NNC 26-9100 (n=10/dose) and (B) Octreotide (n=6/dose). * p<0.05 as compared to vehicle (0 μg), one-way ANOVA.

FIGS. 2A and 2B shows bar graphs of results of evaluation of SAMP8 brain tissues following NNC 26-9100 administration (i.c.v.). (A) ELISA analysis of Allx-42 performed and calculated as pg/ml/gram of brain tissue and normalized to vehicle (n=6/group). (B) Western blot expression analysis of SSTR4, APP, and NEP performed with optical densities normalized to vehicle (0 μg) animals (n=5-6/group). * p<0.05 as compared to vehicle, one-way ANOVA.

FIG. 3 is a bar graph of results when acquisition (learning) by SAMP8 mice was evaluated via T-maze task 24 h following i.c.v. injection of 0.2 μg NNC 26-9100 (NNC) or vehicle (Veh) in the presence of 1 or 10 mM of the NEP inhibitor phosphoramidon (P) (n=6-8/group). * p<0.05, ** p<0.01 as compared to NNC-26 9100 alone, † p<0.05 as compared to NNC+1 mM P, two-way ANOVA.

FIGS. 4A and 4B shows bar graphs of results of evaluation of SAMP8 brain tissues following i.c.v. injection of 0.2 μg NNC 26-9100 (NNC) or vehicle (Veh) in the presence of 1 or 10 mM of the NEP inhibitor phosphoramidon (P). (A) ELISA analysis of Aβx-42 performed and calculated as pg/ml/gram of brain tissue and normalized to NNC 26-9100 alone (n=4-5/group). (B) Western blot expression analysis of SSTR4, APP, and NEP performed with optical densities normalized to NNC 26-9200 alone group (n=5-6/group). * p<0.05 as compared to NNC 26-9100 alone, † p< 0.05 and ‡ p<0.01 as compared to NNC+1 mM P, two-way ANOVA.

FIGS. 5A, 5B and 5C shows bar graphs of results when recognition (memory) of SAMP8 mice was evaluated. (A) Object-recognition task 24 h after initial training period and 48 h after i.c.v. injection of vehicle or NNC 26-9100 (0.2 μg) (n=7/group). (B) ELISA analysis of Aβx-42 performed and calculated as pg/ml/gram of brain tissue and normalized to vehicle (n=5-6/group). (C) Western blot expression analysis of SSTR4, APP, and NEP performed with optical densities normalized to vehicle (n=5-6/group). ** p<0.01 as compared to vehicle (0 μg), Student's t-test.

FIGS. 6A and 6B shows graphs of multiple-time regression analysis of 131I-NNC 26-9100 following intravenous injection. (A) Slope of the line shows unidirectional uptake of blood to-brain, plotted as brain/ratio versus exposure time (EXPT). (B) Percent of injected dose of 131 I-NNC 26-9100 per gram of brain tissue over time.

FIG. 7 shows a graph of kinetics of blood-to-brain transport of 131 I-NNC 26-9100 after i.v. administration, showing clearance of 131 I-NNC 26-9100 from blood after i.v. injection. Inset shows the initial distribution phase was linear.

FIG. 8 is a bar graph showing regional variation in brain/serum ratio determination in mouse brain uptake at 8 min post i.v. injection of radio labled I-NNC 26-9100 (n=5/region). Striatum (St), frontal cortex (FC), hypothalamus (Hypo), parietal cortex (PC), hippocampus (Hip), thalamus (Th), midbrain (Mb), pons medulla (PM), cerebellum (Cb), occipital cortex (OC),whole brain (WBr). *** p<0.001, as compared to whole brain, one-way ANOVA.

FIGS. 9A and 9B shows bar graphs of effects on acquisition learning at day-21 (A), and retention memory at day-28 (B), of 12-month old SAMP8 mice following i.p. injection (0.2-200 μg/day) of NNC 26-9100 (n=8-10/group). *p<0.05; ** p<0.01 (0 ug is vehicle control), one-way ANOVA.

FIG. 10 is a bar graph of results obtained from SAMP8 brain tissues analyzed for % change in 0-amyloidx-42 following NNC 26-9100 administration. ELISA analysis of A1142 performed and calculated as pg/ml/gram of brain tissue and normalized to vehicle (n=6/group).*p<0.05 (0 ug is vehicle control), one-way ANOVA.

FIG. 11 is a bar graph of results obtained with Western blot expression analysis of SSTR4, APP, and NEP performed with optical densities normalized to vehicle (0 μg) animals (n=5-6/group). No significant shown compared to vehicle, one-way ANOVA.

FIGS. 12A and 12B shows bar graphs of effects on acquisition learning at day-21 (A), and retention memory at day-28 (B), of 12-month old SAMP8 mice following i.p. injection (0.2-200 μg/day) of LIU 686-06A (n=8-10/group). *p<0.05; ** p<0.01 (0 ug is vehicle control), one-way ANOVA.

FIG. 13 is a bar graph of effects on avoidance learning acquisition in APPswetransgenic mice following i.c.v. injection (n=3-4/group) of NNC 26-9100.

### DETAILED DESCRIPTION

The present disclosure provides methods for mitigating cognitive decline in subjects, such as patients suffering from Alzheimer's disease (AD). These methods are useful for (i) reducing levels of beta-amyloid protein (A11₄₂) in brain tissue in the subject, and (ii) improving memory function in the subject. The methods are useful in circumstances where cognitive impairment has occurred, or is in the process of occurring, wherein the impairment can be measured by any one or more of a number of standard tests of memory function in the subject.

The somatostatin receptor subtype-4 is (SSTR4) is expressed in relatively high levels in neocortex and hippocampus, which are important for learning and memory, and is also significantly decreased in patients with AD. Yet the precise interactions of the SSTR4 subtype with other SSTR subtypes, effects of its activation on memory function and behavior and specific impact on A11₄₂ levels in brain tissue were not known or predictable. Moreover, the ability of SSTR4 agonists to cross the blood brain barrier and have specific effects in brain tissue was not known. Described herein is the surprising discovery that administration of selected non-peptide SSTR4 agonists not only improves memory function in animal models of AD, but also decreases A11₄₂ levels in brain tissue. Further, the present disclosure reveals that that both acute intracerebral-vascular and chronic intraperitoneal administration of these SSTR4 agonists achieves the observed results. Thus the present disclosure describes the unexpected and useful finding that the selected agonists can be administered peripherally, will cross the blood-brain barrier and will reach those brain areas critically affected by AD. It will be further understood that the present disclosure applies also to other learning and memory disorders arising from other disease processes that affect SSTR4.

In brief, the methods involve administering an SSTR4 agonist to a subject in need thereof, which can be for example a human patient with a diagnosis of AD according to generally recognized clinical standards, or any animal which constitutes an established animal model of AD such as but not limited to the SAMP8 mouse, and the APPswe transgenic mouse. Cognitive function, specifically memory impairment associated with AD, may be determined by a variety of diagnostic tests known in the art, which detect cognitive impairment and specifically memory impairment. Examples of such memory function tests for animal subjects such as rats or mice include maze learning paradigms and object recognition tests such as those described in the Examples herein below. Human subjects can be evaluated before, during and after administration of the SSTR4 agonist using any one of a number of available clinical tests, such as for example the Addenbrooke's Cognitive Examination (ACE), which is a 100-point test battery that assesses six cognitive domains and has been used to reliably differentiate early AD from frontotemporal dementia (FTD). (Mathuranath, P. S. et al., Neurology. 2000 Dec. 12; 55(11):1613-20). Other methods for evaluating memory function in human patients are well known and include mental status examinations that assign a score or index according to the subject's answers to questions including those directed at determining the subject's general orientation (e.g. the time, date, and season; the place where the subject lives, type of building, city and state; the subject's name, age, and occupation); attention span (e.g. in which a qualified tester scores the subject's ability to complete a thought in conversation, or to follow a series of directions); recent memory (e.g. in which a qualified tester asks questions related to recent people, places, and events in the subject's life or in the world); remote memory (e.g. in which the qualified tester asks questions about the subject's childhood, school, or historical events that occurred earlier in life; word comprehension and judgment/logical problem solving (e.g. in response to proposed hypothetical situations). Also suitable for example is a cognitive screening test known as the “TYM test” (J. Brown et al., 338 BMJ 2030, Jun. 9, 2009). Suitable such tests also include any cognitive screening test that evaluates memory performance and in which a score, level or degree of memory performance can be assigned to the subject at the time of the test.

The SSTR4 agonists can be administered to subjects: (i) to treat memory impairment that has already occurred or (ii) to prevent further memory impairment from occurring. In an exemplary embodiment, the subject is a human patient with a clinical diagnosis of AD, which can include positive tests of memory impairment including a specified level or degree of memory impairment.

The SSTR4 agonist is for example a compound of formula I (“NNC 26-9100”):

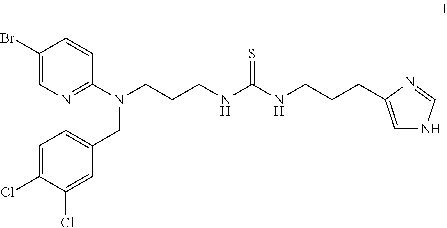

Alternatively the SSTR4 agonist can be a compound of formula II: (“LIU—686-06A”).

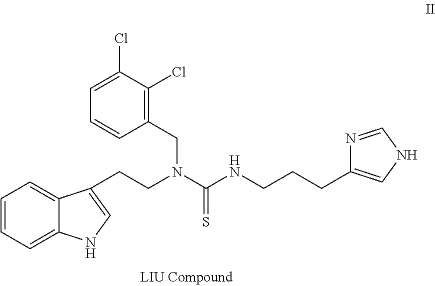

Alternatively the SSTR4 agonist can be a compound of formula III (“3-(3-1H-Imidazol-4-yl)propyl-1-(2-(1H-indol-3-yl)ethyl-1-(3,4-dichlorobenzyl)urea”):

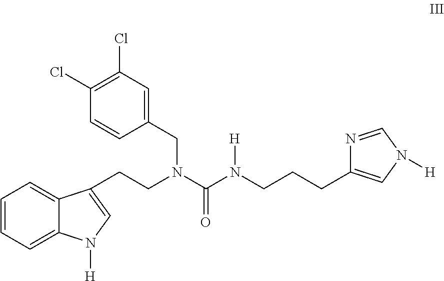

The compounds of formulae I and II are described in, and can be prepared by one or more of the synthetic methods described in U.S. Pat. Nos. 6,020,349, issued Feb. 1, 2000, and 6,083,960, issued Jul. 4, 2000, and in A. M. Crider et al., Letters in Drug Design and Discovery 1:84-87 (2004), the entire disclosures of which are herein incorporated by reference. Formula I is the molecular structure of selective somatostatin receptor subtype-4 agonist “NNC 26-9100”, having a Ki=6 nM at SSTR4 and an EC50=26±6 nM. Formula II is the molecular structure of the selective somatostatin receptor subtype-4 agonist “LIU 686-06A”. Formula III is the molecular structure of the novel compound 3-(3-1H-Imidazol-4-yl)propyl-1-(2-(1H-indol-3-yl)ethyl-1-(3,4-dichlorobenzyl)urea that can be synthesized by the method described in Example 4 herein below.

The amount of SSTR4 agonist to be administered to a subject that is a human patient, and that constituting a “therapeutically effective amount” will generally depend on the specific circumstances of the patient, including among other factors the patient's sex, weight, age, and general health, as well as the severity of cognitive decline in the patient. The effective amount of SSTR4 agonist will typically be determined by an attending physician using such information in combination with the published results of basic and clinical studies on the particular compound. For example, a therapeutically effective amount of a SSTR4 agonist will range for example from about 0.01 mg/kg/day to about 10 mg/kg/day. An exemplary dosage range is suitably 0.1-1000 mg/day, or in an exemplary embodiment 1-500 mg/day, which can be administered in a once-daily dosage form or distributed across several smaller dosages during the day, such as in dosages formulated to deliver the entire daily dose in two, three or four dosages. Daily dosages outside the stated range may be required depending on the specific circumstances of the particular patient being treated, and the specific SSTR4 agonist being administered. Variations on the dosage may be determined by the attending physician in view of the particular circumstances, and the responsiveness of the subject to a particular dosing regimen may be evaluated using the memory function tests as described elsewhere herein.

Routes of administration include those through which peripheral administration is achieved. The term “peripheral” refers to administration via a vein, muscle or artery that is not located within the trunk of the body, i.e. not in the chest or abdomen. Suitable peripheral access sites include for example veins of the arms and legs. Peripheral administration can be achieved by normal routes of administration including parenteral routes, to achieve systemic distribution of the SSTR4 agonist and delivery to brain tissue. Parenteral routes of administration can include injection or infusion, as by intravenous, intraarterial, intramuscular, intracerebral, i ntracereb rove ntricu la r, subcutaneous, and intraperitoneal by infusion or injection. Alternatively, enteral routes of administration can be used and include oral, as by tablets, capsules, or drops; gastric as by feeding tube; and rectal in suppository or enema form.

The SSTR4 agonist will generally be administered in the form of a pharmaceutical composition further comprising a pharmaceutically acceptable carrier, excipient or diluent as known in the art. Such compositions are generally formulated in a conventional manner utilizing solid or liquid vehicles or diluents as appropriate to the mode of administration. For purposes of parenteral administration, solutions of the SSTR4 agonist that are used in the methods of the present invention are formulated according to standard techniques. For example, solutions of an SSTR4 agonist can be made in isotonic saline, or lactated Ringer's solution, which can include an amount (e.g. 1%) of bovine serum albumin. The aqueous solutions should be suitably buffered if necessary and the liquid diluent first rendered isotonic. These aqueous solutions are suitable for intravenous injection purposes. Oil-based solutions or suspensions can be made for intra-articular, intramuscular and subcutaneous injection purposes. The preparation of all liquid formulations under sterile conditions is readily accomplished by standard pharmaceutical techniques well known to those skilled in the art.

For purposes of oral administration, tablets containing excipients such as sodium citrate, calcium carbonate, and di-calcium phosphate may be employed, along with various disintegrants such as starch, preferably potato or tapioca starch, alginic acid and certain complex silicates, together with binding agents such as polyvinyl pyrrol idone, sucrose, gelatin and acacia. Additionally, lubricating agents such as, but not limited to, magnesium stearate, sodium lauryl sulfate and talc are often very useful for tableting purposes. Solid compositions of a similar type may also be employed as fillers in soft elastic and hard filled gelatin capsules. Preferred materials in this connection also include by way of example lactose or milk sugar as well as high molecular weight polyethylene glycols. When aqueous suspensions and/or elixirs are desired for oral administration, the essential active ingredient may be combined with various sweetening or flavoring agents, coloring matter or dyes and, if so desired, emulsifying and/or suspending agents, together with diluents such as water, ethanol, propylene glycol, glycerin and various combinations thereof.

The methods encompass improving memory function in a subject suffering by first evaluating the subject for memory function, i.e. determining a first level of memory function in the subject, and administering to the subject a therapeutically effective amount of a somatostatin receptor-4 (SSTR4) agonist. Following administration, the patient is re-evaluated for memory function, i.e. a second level of memory function is determined. The first and second memory function levels are determined according to laboratory or clinical tests as described elsewhere herein. The first and second levels of memory function in the subject, such as a first and a second test score, are compared. A change in the level of memory function, such as a change in test scores between the first evaluation and the second evaluation indicates whether the subject is responding to the administration of the SSTR4 agonist. For example, a second test score that is better than a first test score indicates that the subject is positively responding to the administration of the SSTR4 agonist.

In another aspect, the present disclosure provides a method to improve memory function in a subject suffering from Alzheimer's disease, the method comprising reducing AP42 levels in brain tissue of the subject by administering to the subject a therapeutically effective amount of an a somatostatin receptor-4 (SSTR4) agonist. AP42 levels in brain tissue of the subject, in the case of animal subjects can be readily evaluated by any one of a number of known ex vivo assays including immunoassays such as ELISA. In human subjects, a reduction in AP42 in the brain is inferred, based on the results described herein, from an improvement in memory function as determined from cognitive testing.

The following Examples are offered for the purpose of illustrating the present disclosure and are not to be construed as limiting of the scope of the claims. The disclosures of all references cited herein are hereby incorporated by reference in their entireties.

### Example 1

### Effects of Acute Administration of NNC 26-9100 (i.c.v.) on Acquisition (Learning) and Recognition (Memory)

This investigation evaluated the selective and stable SSTR4 agonist NNC 26-9100 (Formula I), a non-peptide drug having a >100-fold selectivity for SSTR4 over the other SSTR subtypes (see, e.g., Crider et al., 2004). The effects of NNC 26-9100 (i.c.v.) on acquisition (learning) and recognition (memory) were evaluated using the senescence accelerated mouse prone-8 (SAMP8) model of cognitive decline. The SAMP8 mouse strain undergoes agedependent learning and memory deficits with increased amounts of APP and soluble All in brain tissue similar to those observed in AD (Kumar et al., 2000; Morley et al., 2002; Poon et al., 2004; Tomobe and Nomura, 2009). Furthermore, at 12-months of age, SAMP8 mice do not express plaques allowing the examination to focus on the soluble form of All implicated in hippocampal learning and memory behavior (Selkoe, 2008). To investigate NNC 26-9100 NEP dependent activity, the NEP inhibitor phosphoramidon was co-administered with NNC 26-9100.

Following respective learning and memory assessments, brain tissue analyses were performed ex vivo to determine drug effect on All42 levels and expression of NEP, SSTR4 and APP.

Materials and Methods: Chemicals.

NNC 26-9100 was synthesized, purified, and confirmed via NMR by Dr. A. M. Crider per previously established protocols (Ankersen et al., 1998; Crider et al., 2004). All other chemicals and reagents, unless otherwise stated, were purchased from Sigma-Aldrich (St. Louis, Mo.).

Animals.

Twelve-month old male SAMP8 mice were used for all behavioral and post-treatment molecular assessments. Mice were housed in rooms with a 12 h light/dark cycle (20-22° C.) with water and food available ad libitum. All experiments were conducted in accordance with the institutional approval of the animal use subcommittee, which subscribes to the NIH Guide for Care and Use of Laboratory Animals. SAMP8 mice were obtained from the breeding colony at the Veterans Affairs Medical Center—VA hospital (St. Louis, Mo.). The colony is derived from siblings generously provided by Dr. Takeda (Kyoto University, Japan).

T-Maze Testing. The T-maze avoidance apparatus training and testing procedures have been previously described, and shown as an effective means to assess learning in SAMP8 mice (Flood and Morley, 1993; Farr et al., 2000). Measurement of the effects of NNC 26-9100 or octreotide on acquisition was performed following injection (i.c.v.) in 12-month-old male SAMP8 mice. Octreotide is a primarily SSTR2 agonist, of the SRIF-1 family, serving as an additional control set. Forty-eight hours prior to testing, the mice were anesthetized with 2,2,2-tribromoethanol, placed in a stereotaxic instrument, and the scalp was deflected. A unilateral hole was drilled 0.5 mm anterior and 1.0 mm to the right of the bregma. Twenty-four hours prior to training, the mice were again placed under light anesthesia, and received an injection of NNC 26-9100 or octreotide (0, 0.002, 0.02, 0.2, or 2.0 μg). This time frame is consistent with work by others showing somatostatin administration over 24 hrs decreased Aβ1-42 concentrations in the brain (Saito et al., 2005). The NNC 26-9100 dose identified to induce the greatest effect (0.2 μg) was then evaluated against NEP inhibitor phosphoramidon (1 or 10 mM) (American Peptide Co, Sunnyvale, Calif.). Twenty-four hours following drug administration mice were evaluated using the T-maze test.

The T-maze consisted of a black plastic alley with a start box at one end and two goal boxes at the other. The start box was separated from the alley by a plastic guillotine door, which prevented movement down the alley until training began. An electrifiable stainless steel rod floor ran throughout the maze to deliver scrambled foot-shock. Mice were trained and tested between 07:00 and 15:00 h. Mice were not permitted to explore the maze prior to training. A block of training trials began when a mouse was placed into the start box. The guillotine door was raised and a buzzer sounded simultaneously; 5 s later foot-shock was applied. The goal box entered on the first trial was designated “incorrect” and the foot-shock was continued until the mouse entered the other goal box, which in all subsequent trials was designated as “correct” for the particular mouse. At the end of the acquisition trial, mice were decapitated and the brains flash frozen and stored at −80° C. for subsequent analyses.

Object-Recognition Testing.

Object-recognition is a non-spatial memory recognition task, shown to be effective in SAMP8 mice (Bevins and Besheer, 2006; Fontan-Lozano et al., 2008). In this task, treated and control mice are measured as to the difference in time spent with a familiar (or remembered) object and a novel object. Mice received a single optimized dose (0.2 μg) determined from T-maze assessment, against vehicle control (0 μg). Dosing was performed 24 h prior to the initial training period and the recognition task was evaluated 24 h after training (i.e. 48 h after dosing). Mice were habituated to an empty apparatus (55×40×40 cm) for 5 min a day for 3 days prior to entry of the objects. On the day of training two similar objects were placed in the maze. Mice were placed in the maze and allowed to explore the objects for 5 min. In the 24-h retention test, one of the same objects is placed in the maze, as well as a new object in a new location. The difference in percent time spent exploring the new object over the familiar object was then determined. The criteria for exploration were based strictly on active exploration, where the mouse had both forelimbs within a circle of 1.5 cm around the object, with its head oriented toward it, or when touching it with its vibrissae. At the end of the retention trial, mice were decapitated and the brains flash frozen and stored at −80° C. for subsequent analyses.

Evaluation of Aβ.

Brain levels of Aβ42 were evaluated ex vivo, from flash frozen whole brain tissue following respective behavioral analyses, using ultra-centrifugation and solid phase extraction methods (Lanz and Schachter, 2006; Zupa-Fernandez et al., 2007), coupled with Enzyme-linked immunosorbent assay (ELISA) analysis. Half of the brain tissue was dedicated to Western blot examinations with remainder used for Aβ42 evaluation. Brain tissue was homogenized and incubated with 1 mL 50 mM NaCl, 0.4% DEA, pH=10, containing protease inhibitor (Roche, Indianapolis, Ind.). Samples were kept on ice for 20 min, followed by sonication at 30% for 30 sec. Samples were then incubated at room temperature (3 h) followed by centrifugation at 355,000×g for 30 min (4° C.). Supernatant was collected for extraction using Oasis HLB 3 cc columns (Waters, Milford, Mass.). Oasis columns were activated with 2 mL methanol (MeOH), followed by 2 mL diH2O. Brain homogenates were loaded in 1 mL increments. Samples were then washed sequentially with 1 mL volumes of 5% and 30% MeOH, then eluted with 1 mL 2% NH4OH in 90% MeOH. Eluted samples were collected and vacuum-centrifuged at 1400 rpm, 60° C. for 90-120 min. Once samples were dried completely, they were stored at −80° C. until assay. Samples were reconstituted and analyzed in duplicate according to ELISA assay directions (Beta-Mark Aβx-42, Covance, Dedham, Mass.) via luminometer (Lumistar Optima, BMG Labtech, Durham, N.C.). Aβ42 levels were calculated from linear-regression curve in pg/ml and then set to gram weight of brain tissue and normalized to respective controls.

Protein Expression.

Protein expression of brain tissues was evaluated ex vivo, from flash frozen whole brain tissue following respective behavioral analyses, by Western blot analyses for SSTR4, APP and NEP. Tissues were homogenized in RIPA buffer containing protease inhibitor (Roche) using a 1:10 ratio (0.5 g=5 ml), transferred into tubes and spun at 10,000×g for 20 min (4° C.). The supernatant was taken and a protein assay was used to determine the amount of protein present. Samples (100 μg) were separated using an electrophoretic field on Biorad (Bio-Rad, Hercules, Calif.) Tris HCl gels (10%) at 175 V for 70 min following heating at 95° C. for 5 min. GelCode Blue Stain (Pierce, Rockford, Ill.) was used to confirm appropriate protein loading. The proteins were then transferred to nitrocellulose membranes with 240 mA at for 45 min (4° C.). The membranes were then blocked using 5% nonfat milk-Tris-buffered saline (20 mM Tris base, 137 mM NaCl, pH 7.6) with 0.1% Tween-20 for 4 h and then incubated overnight at 4° C. with primary antibodies (1:1,000-1:2,000 dilution) in PBS-0.5% BSA. Antibodies shown effective: SSTR4 (Sigma-Aldrich), NEP (Millipore, Billerica, Mass.), and APP (Millipore). The membranes were then washed with 5% nonfat milk-Tris-buffered saline buffer before incubation with the respective secondary antibody at a 1:2,000 dilution (in PBS-0.5% BSA) for 60 min at room temperature. Blots were developed using the enhanced chemiluminescence method (ECL+; Amersham Life Science Products; Springfield, Ill.), and protein bands visualized on X-ray film. Optical densities were measured by densitometer (Biorad) and evaluated against respective controls. After each initial protein expression assessment, membranes were then stripped and re-probed with actin (Sigma-Aldrich).

Statistical Analyses.

Comparisons of multi-group data were made using one-way or two-way ANOVA as appropriate, with Newman-Keuls post-hoc analysis. Student's t-test was used when only two groups were evaluated. Data are expressed as means±SEM.

Results: NNC 26-9100 Dosing-Range Impact.

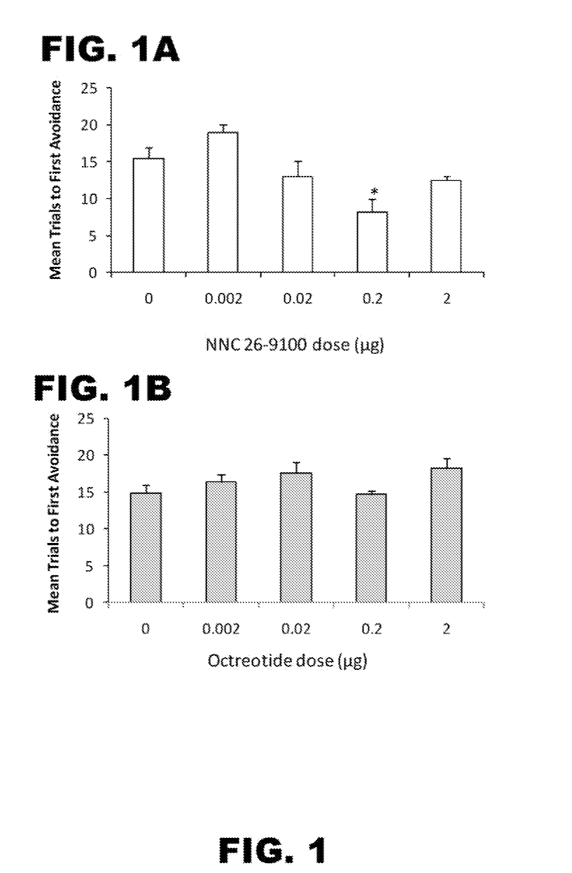

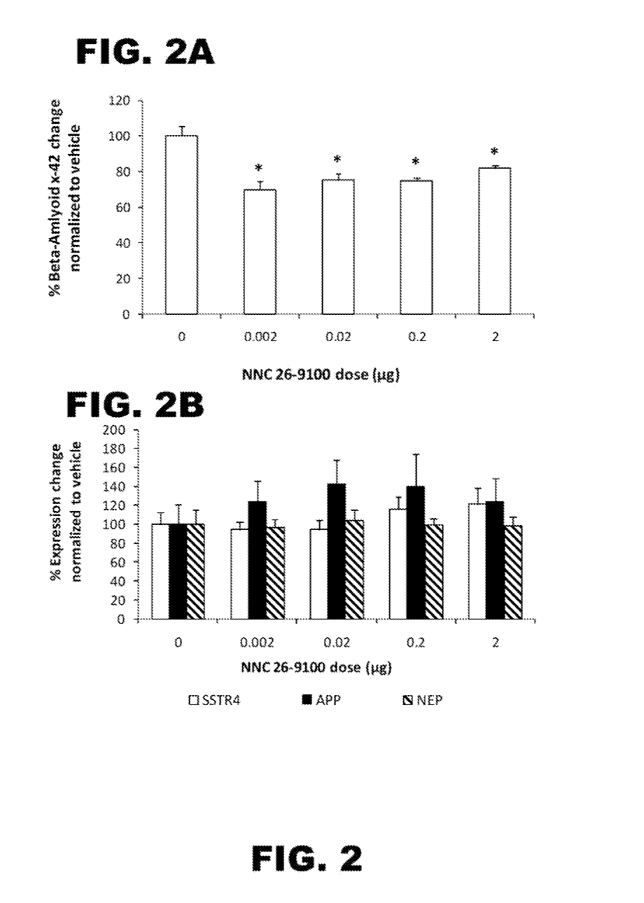

Acquisition learning in SAMP8 mice was conducted via the T-maze foot-shock avoidance test following i.c.v. administration of NNC 26-9100 (0.002-2.0 μg, with 0 μg vehicle control). NNC 26-9100 at 0.2 μg showed a significant improvement (p<0.05) in learned avoidance, indicated by the lower number of mean trials compared to vehicle controls (n=10/dose) (FIG. 1A). In a separate experiment, mice were treated with the SSTR2 agonist, octreotide to determine its impact on learned acquisition over an identical dosing range (0.002-2.0 μg). In mice treated with octreotide, no significant effects on learned acquisition were found at any dose compared to vehicle control (FIG. 1B). ELISA analysis of Aβ42 on extracted brain tissues of SAMP8 mice was conducted after learning and memory evaluations. Post-T-maze evaluation, a significant decrease (p<0.05) of ˜20% in Aβ42 was shown across all doses of NNC 26-9100 (0.002-2.0 μg) compared to vehicle (n=6) (FIG. 2A). Western blot analyses found no significant changes in the expression of SSTR4, APP, or NEP in mice treated with NNC 26-9100 (0.002-2.0 μg) when compared to vehicle control (n=5-6/group) (FIG. 2B). Actin expression levels were consistent across all respective evaluation sets.

NNC 26-9100 and Phosphoramidon Coadministration.

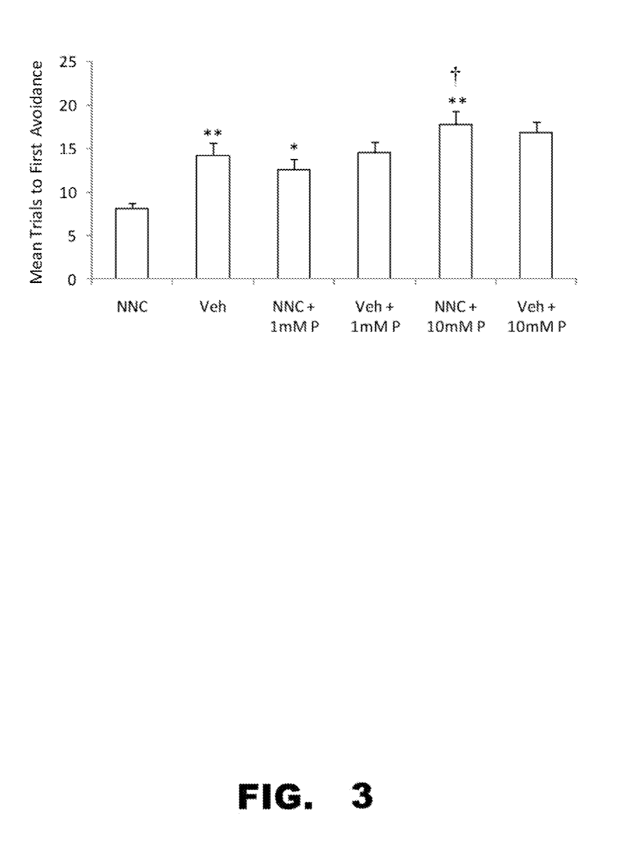

The NNC 26-9100 dose identified to induce a significant increase in acquisition learning (0.2 μg) was then evaluated against the NEP inhibitor phosphoramidon (1 or 10 mM, co-administered with NNC 26-9100) in the T-maze test (n=6-8) (FIG. 3). Mice treated with NNC 26-9100 alone showed a significantly lower number of mean trials to first avoidance (p<0.01), demonstrating an improved learned avoidance over vehicle control. Mice treated with NNC 26-9100+1 mM phosphoramidon (p<0.05) or NNC 26-9100+10 mM phosphoramidon (p<0.01) showed a significant increase in the number of mean trials to first avoidance over NNC 26-9100 alone treated group, demonstrating that NEP inhibition decreased learning. Additionally, mice treated with NNC 26-9100+10 mM phosphoramidon demonstrated a significantly higher number of trials to first avoidance and decreased acquisition when compared to mice treated with NNC 26-9100+1 mM phosphoramidon (p<0.05), demonstrating that NEP blocked the beneficial effects of NNC 26-9100 on learning. No significant change was observed in the number of mean trials to first avoidance between mice treated with vehicle or vehicle combined with any dose of phosphoramidon.

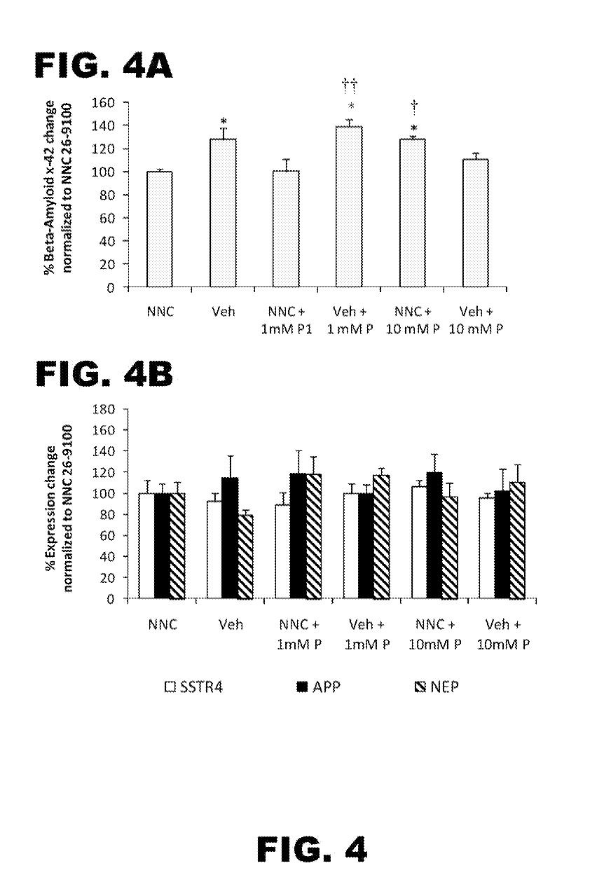

ELISA analysis of Aβ42 was also evaluated from brain tissues within the NNC 26-9100 (0.2 μg) and phosphoramidon evaluation, post-T-maze evaluation (n=4-5/group) (FIG. 4A). Both vehicle group and NNC 26-9100+10 mM phosphoramidon showed a significant increase (p<0.05) over the NNC 26-9100 alone group. Additionally, the vehicle+1 mM phosphoramidon group showed a significant increase over the NNC 26-9100+1 mM phosphoramidon group (p<0.05). No change within vehicle groups (vehicle alone, +1 mM or +10 mM phosphoramidon) was shown.

No changes were shown in SSTR4, APP, or NEP within NNC 26-9100 alone and coadministered phosphoramidon (+1 mM or +10 mM) groups, or between vehicle alone and NNC 26-9100 alone groups (n=5-6/group) (FIG. 4B). Actin expression levels were consistent across all samplings within respective evaluation sets.

Object-Recognition Evaluation.

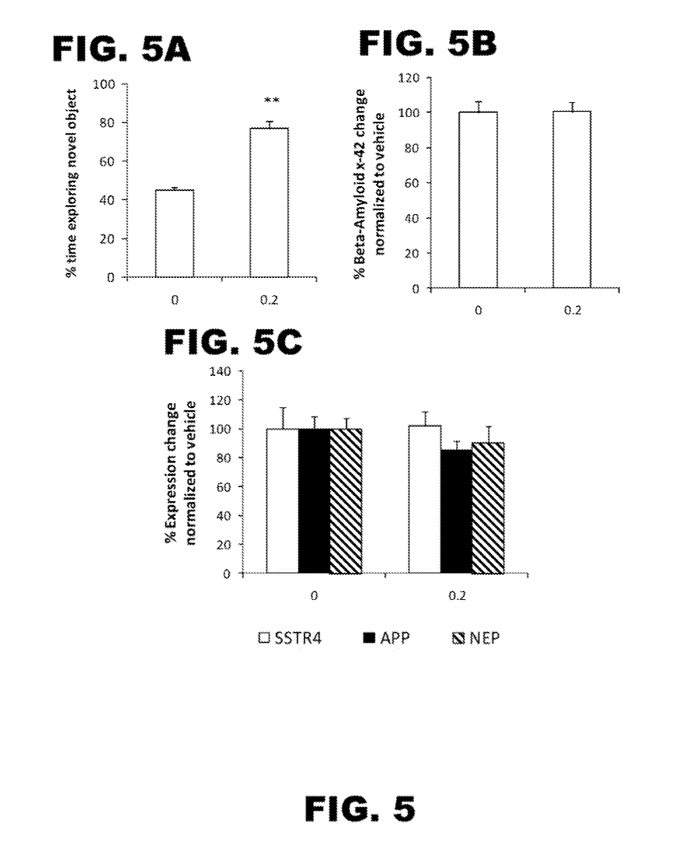

NNC 26-9100 (0.2 μg) was then evaluated for effects on recognition (memory) in SAMP8 mice in the object-recognition task (n=7/group) (FIG. 5A). Mice treated with NNC 26-9100 spent significantly more time with the novel object versus the non-novel object when compared to vehicle (p<0.01) showing an approximately 2-fold enhancement in recognition memory.

ELISA analysis of Aβ42 showed no significant change between vehicle (0 μg) and NNC 26-9100 (0.2 μg) treated animals (FIG. 5B). No significant changes were found in the expression of SSTR4, APP, or NEP between vehicle or NNC 26-9100 (0.2 μg) treated animals (FIG. 5C). Actin expression levels were consistent across all samplings within respective evaluation sets.

### Example 2

### Effects of Chronic Peripheral Administration NNC 26-9100 (i.p.) on Acquisition (Learning) and Recognition (Memory)

Materials and Methods: Chemicals.

NNC 26-9100 was obtained as described in Example 1. All other chemicals and reagents, unless otherwise stated, were purchased from Sigma-Aldrich (St. Louis, Mo.).

Animals.

Twelve-month old male SAMP8 mice were used for all behavioral and post-treatment molecular assessments. Male CD-1 mice were used for assessments of brain uptake and distribution. Mice were housed in rooms with a 12 h light/dark cycle (20-22° C.) with water and food available ad libitum. All experiments were conducted in accordance with the institutional approval of the animal use subcommittee, which subscribes to the NIH Guide for Care and Use of Laboratory Animals. SAMP8 mice were obtained from the breeding colony as in Example 1 above.

Labeling.

The chloramines-T method was use to radioactively label NNC 26-9100, with 5 μg added to 2 mCi of 131I (PerkinElmer Life and Analytical Sciences, Shelton, Conn.) along with 10 μl of chloramine-T. Radioactively labeled NNC 26-9100 was purified on a Sephadex G-10 column.

Brain Influx Rate and Serum Clearance.

Mice were anesthetized with an i.p. injection of urethane (40% solution). The right carotid artery and left jugular vein were exposed. Mice were injected with 300,000 cpm labeled 131I-NNC 26-9100 in a volume of 200 μl lactated Ringer's solution containing 1% bovine serum albumin by injection into the jugular vein. Mice were maintained under a heat lamp before and after the i.v. injection and monitored for respiratory difficulty. Blood was collected from the right carotid artery, and the whole brain (WBr) was removed and weighed immediately at 1, 2, 3, 4, 5, 6, 7, 8, 9, 10, 15, 30, or 45 min after the jugular injection. Blood was centrifuged at 5400×g for 10 min (4° C.), and the level of radioactivity was measured from 50 μl of the resulting serum. The level of radioactivity was also counted in a gamma counter. Brain/serum ratios (μl/g) were determined and plotted against exposure time (Expt).

The brain/serum ratio (μl/g) was calculated as follows:

$$Brain \text{/} serum = \frac{cpm brain}{( cpm \text{/} µl serum ) ( brain weight )}$$

And Expt in minutes was calculated as follows:

$$Expt = {\int }_{0}^{t} \frac{{C}_{p} ( t ) d t}{{C}_{p} t}$$

where Cp is cpm per ml of serum, t is time in minutes and Cpt is the cpm in the serum at time t.

When the brain/serum ratio of radioactivity of 131 I-NNC 26-9100 is plotted against the exposure time, the slope of the linear regression line represents the unidirectional influx rate (Ki) of 131I-NNC 26-9100 from blood-to-brain (Patlak et al., 1983). To evaluate clearance from serum, results (cpm in serum) from the brain influx study above were expressed as the percent of the injected dose in each milliliter of serum (% Inj/ml). These values were plotted on a graph against their respective time points (min). The % Inj/ml was calculated from the following formula:

$$\% Inj \text{/} ml = 100 \times \frac{cpm \text{/} ml serum}{cpm injection}$$

The percent of the i.v. injected dose taken up per g of brain (% Inj/g) was calculated for each time point by the equation:

% Inj/g=(brain/serum ration−12)(% Ing/ml)/1000

Capillary Depletion.

Capillary depletion as modified for use in the mouse (Triguero et al., 1990; Banks et al., 2009) was used to determine the degree to which 131 I-NNC 26-9100 was sequestered and retained by the vascular bed of the brain. Mice were anesthetized with urethane and given an injection into the jugular vein of 0.2 ml saline containing 750,000 cpm of 131 I-NNC-26-9100. Single optimized time point (8 min) was used. Arterial blood was obtained, with remaining blood washed-out of the brain by injecting 20 ml of lactated Ringer's solution into the left ventricle of the heart in 60 s. Brain was removed and emulsified in a glass homogenizer (13 strokes) at 4° C. in a 9-fold volume of physiological buffer (10 mM HEPES, 141 mM NaCl, 4 mM KCl, 2.8 mM CaCl₂, 1 mM MgSO4, 1 mM NaH2PO4, and 10 mM D-glucose adjusted to pH 7.4). Dextran solution was added to the homogenate to a final concentration of 26%. An aliquot was centrifuged at 5400×g for 15 min at 4° C. in a swinging bucket rotor. The pellet containing the brain microvessels and the supernatant containing the brain parenchyma were carefully separated. Results were expressed as capillary/serum and parenchyma/serum ratios.

Regional distribution. For regional brain tissue distribution, mice were anesthetized and injected with 131 I-NNC 26-9100, with blood collection followed by decapitation as addressed above. Single optimized time point (8 min) was used. The brain was dissected into regions: frontal cortex, parietal cortex, temporal cortex, occipital cortex, pons medulla, occipital cortex, hippocampus, thalamus, striatum, and cerebellum. The brain region/serum ratio was calculated for each brain region in units of μl/g, as addressed above.

T-Maze testing. The T-maze avoidance apparatus, training and testing procedures were as previously described in Example 1. Measurement of the effects of NNC 26-9100 on acquisition learning and retention was performed following chronic i.p. administration in 12-month-old male SAMP8 mice. Dosing range for i.p. administration (0.2-200 μg) was extrapolated from previous i.c.v. administration data as described in Example 1 above. Respective doses were given once a day over a period of 28 days. Learning and memory was assessed by the T-maze model. Learning evaluations were conducted at three weeks of treatment (day 21), while memory was assessed after one additional week of treatment (day 28). Body weights were evaluated weekly, with no animals in the study exhibiting weight loss or abnormal behaviors. At the end of the evaluations mice were decapitated and the brains flash frozen and stored at −80° C. for subsequent analyses.

Evaluation of Aβ. Brain levels of Aβ42 were evaluated ex vivo as described above in Example 1.

Protein Expression.

Protein expression in brain tissues was evaluated ex vivo as described above in Example 1.

Statistical Analyses.

Blood-to-brain uptake and clearance regression lines were calculated by the least-squares method (Prism 5.0, GraphPad Software Inc. San Diego, Calif.). Comparisons of brain distribution and respective dosing range data were made using one-way ANOVA and Newman-Keuls post-hoc analysis, with data expressed as means±SEM.

Results: Brain Influx Rate and Serum Clearance.

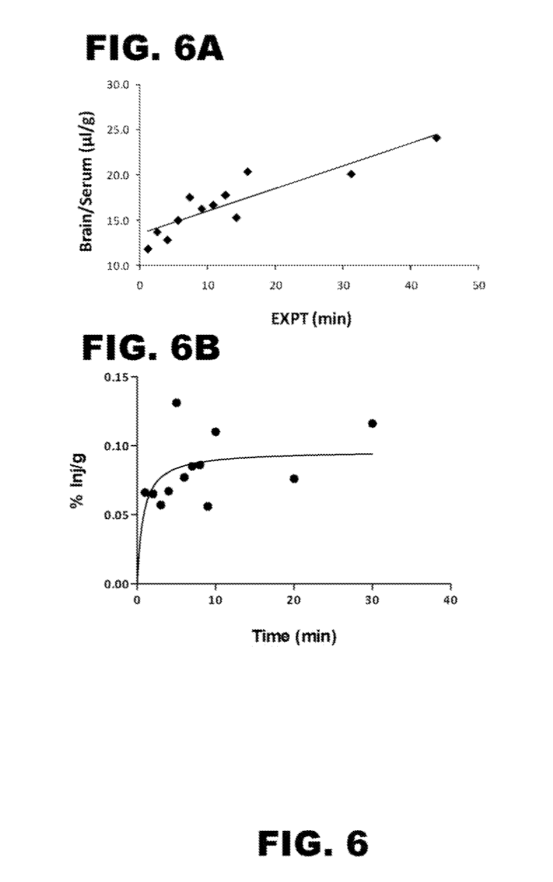

131I-NNC 26-9100 was injected i.v. and evaluated for brain uptake (n=12). FIG. 6A shows the relation between the brain/serum ratios (μl/g) and exposure time (min). The slope of the line represents the rate of influx from blood to brain with a Ki (unidirectional influx rate)=0.25 μl/g min (r=0.899, p<0.0001) (FIG. 1A). The y-intercept representing the initial volume of distribution (Vi) in the brain at time zero=13.46±0.69 μl/g, approximating the vascular space of the brain. The percent of the i.v. dose taken up per g of brain showed a hyperbolic relation with a maximal value calculated as 0.096% Inj/g (FIG. 6B).

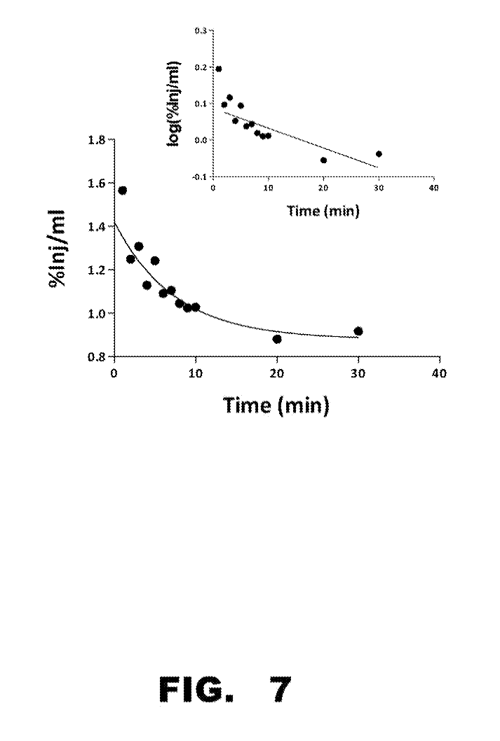

The early phase of clearance from serum after i.v. injection for 131 I-NNC 26-9100 followed first-order kinetics. The relation between the log of levels of radioactivity in arterial serum was expressed as the % Inj/ml versus time after i.v. injection (FIG. 7). The value for 1 min is shown in FIG. 1, but was not used in the calculation of half-time as it likely represent the early distribution phase. Linear regression analysis showed a statistically significant relation between log(% Inj/ml) and time (min) (r=0.841, p<0.01) (FIG. 7, inset). The half-time disappearance rate from serum was 55.5 min.

Regional Distribution.

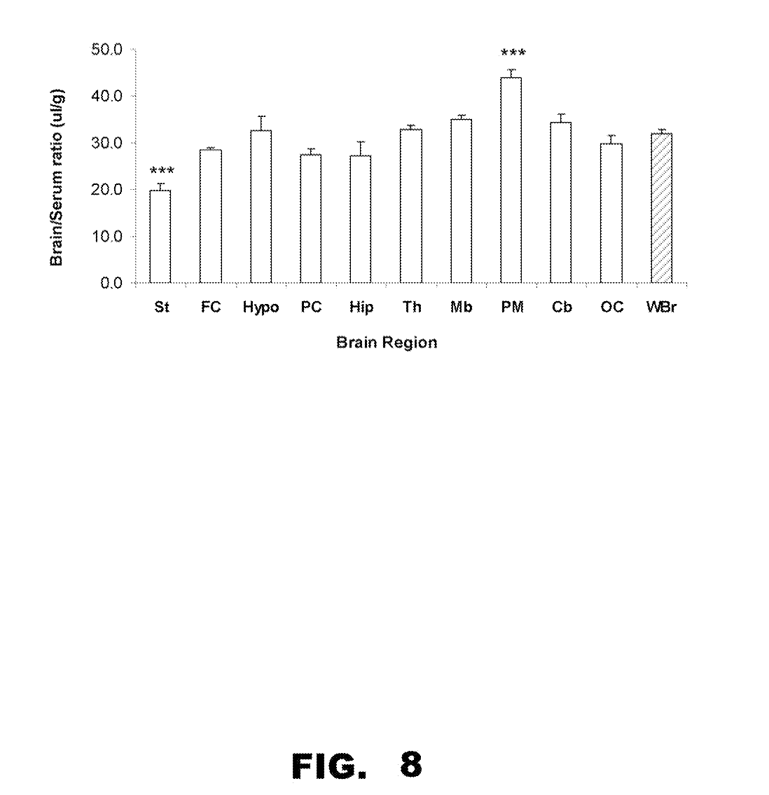

Regional brain uptake evaluation of 131I-NNC 26-9100 was determined at 8 min (period of optimal brain uptake) following i.v. injection (n=5). Data identified I-NNC 26-9100 to have consistent uptake across brain regions, with a significant increase (p<0.001) in the pons medulla and a decrease (p<0.001) in the striatum as compared to whole brain levels (brain/serum ratio in μl/g) (FIG. 8).

Capillary Depletion.

Capillary depletion analysis was conducted to evaluate the proportion of 131I-NNC 26-9100 associated with the brain capillaries (n=5). Results shown 7.2% of 131 I-NNC 26-9100 was associated with the brain capillaries with washout of vascular space at 8 min after i.v. injection.

NNC 26-9100 Learning and Memory.

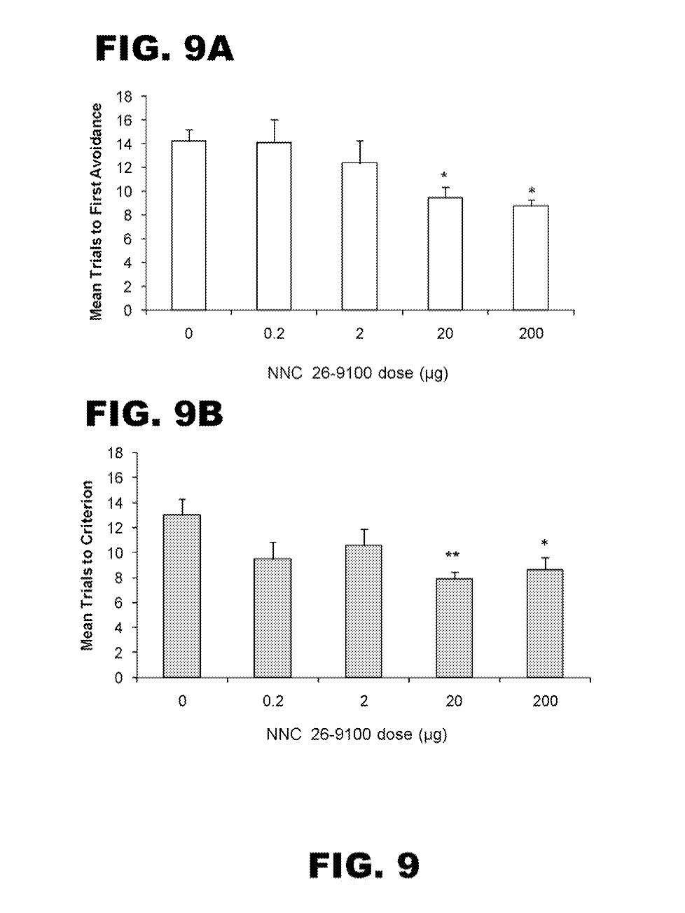

The t-maze was used to test for the effects of chronic NNC 26-9100 on learned acquisition and retention in 12 month SAMP8 mice. On day 21 of treatment, there was a dose dependent decrease in the mean trials to first avoidance. Both the 20 and 200 μg daily doses showed a significantly lower number of mean trials to first avoidance (p<0.05) compared to vehicle control, demonstrating an improvement in learning (FIG. 9A).

Memory was subsequently evaluated in same animals seven days later (on treatment day 28). Mice treated with either the 20 μg (p<0.01) or 200 μg (p<0.05) dose of NNC 26-9100 demonstrated significantly lower mean trials to criterion compared to vehicle treated animals, demonstrating both these doses improved memory (FIG. 9B).

NNC 26-9100 Effect on Aβ42.

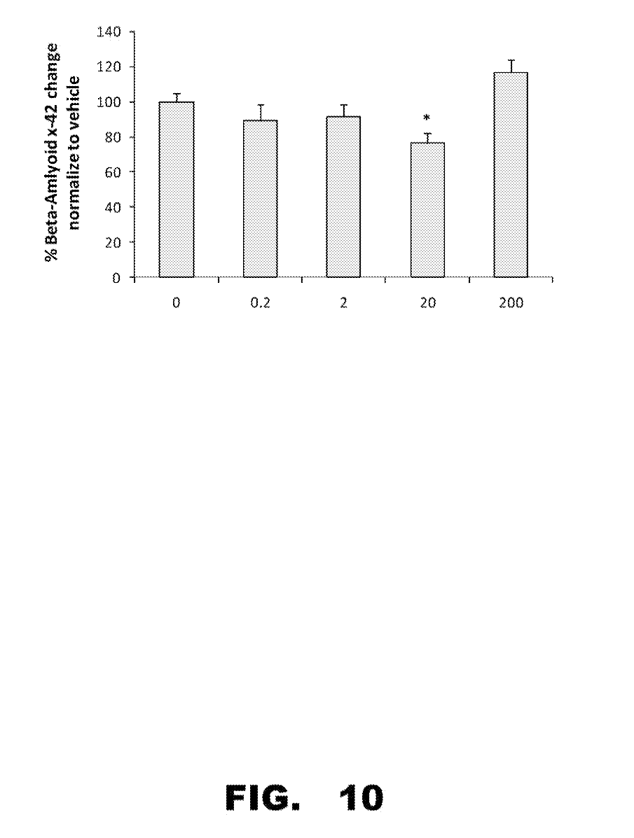

To determine the impact of chronic NNC 26-9100 treatment on Aβ42 levels, ELISA analysis of brain tissues from animals used in the learning and memory experiments were performed. Animals treated with 20 μg of NNC 26-9100 showed a significant decrease (p<0.05) in Aβ42 when compared to vehicle treated animals (FIG. 10). However, no significant changes were observed with any other dose of NNC 26-9100 when compared to vehicle control.

NNC 26-9100 Effect on Protein Expression.

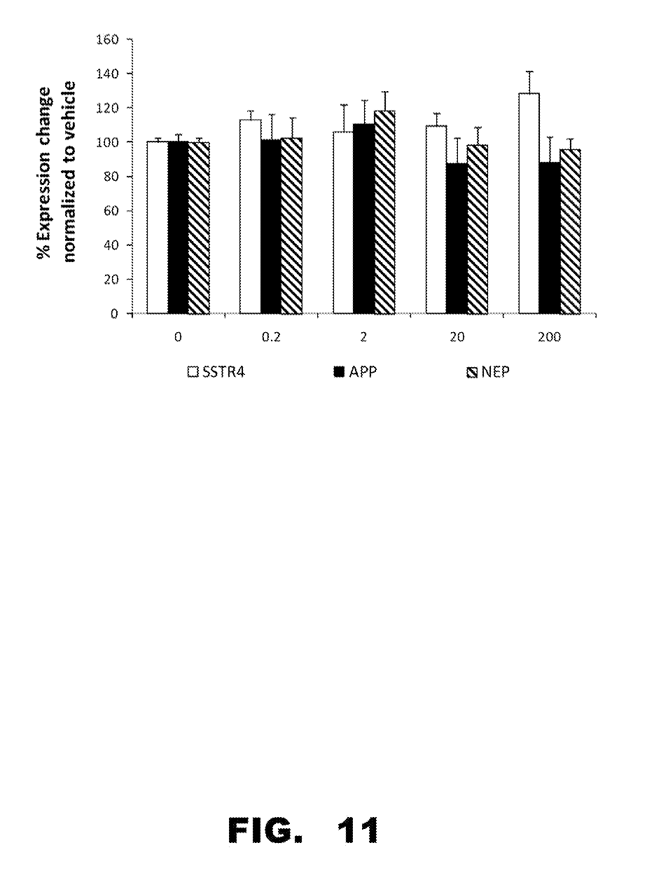

To determine the impact of chronic NNC 26-9100 treatment on expression of SSTR4, APP and NEP, Western blot analyses were performed using brain tissues from animals used in the learning acquisition and retention experiments. No significant changes were observed in the expression of SSTR4, APP, or NEP with any dose of NNC 26-9100 when compared to vehicle control (FIG. 11). Actin expression levels were consistent across all samplings within respective evaluation sets.

The degree to which radioactively labeled NNC 26-9100 crossed from blood-to-brain was thus determined using multiple-time regression analysis. The unidirectional influx-rate (Ki=0.25 μl/g min) identified a moderate degree of uptake. This rate is indicative of non-saturable transport across the BBB, which is dependent on molecular weight and lipophilicity (Oldendorf, 1974) and is consistent based on the chemical characteristics of NNC 26-9100 (i.e. MW: 556, c Log P=5.59). The percent of the i.v. administered dose entering the brain was 0.096% Inj/ml and is well within the range of other therapeutics that exert their effects in brain. The half-time disappearance from serum of 55.5 min falls in viable range for therapeutic use.

The distribution of 131 NNC 26-9100 within the capillary and parenchymal compartments of the brain showed that ˜93% of the compound was associated with the parenchyma. This indicates a limited sequestration of the compound within the capillary component, and that the calculated brain uptake was not an artifact of excessive capillary binding. Thus, a significant portion of NNC 26-9100 is able to actively interact with receptors in the brain.

Regional brain distribution of 131 NNC 26-9100 demonstrated generally uniform uptake. Exceptions to this included the pons medulla and the striatum, showing a significant increase and decrease respectively in uptake compared to whole brain. While the striatum has been shown to be involved in memory selection strategies (Gastambide et al., 2009), other critical regions associated with memory and learning (i.e. cortex, hippocampus), corresponding to SSTR4 receptor distribution (Bruno et al., 1992; Moller et al., 2003), are shown to have similar brain/serum ratios to that of whole brain uptake. Based on uptake rates peripherally administered NNC 26-9100 will distribute to proposed regions of effect.

Chronic NNC 26-91001.p. dosing identified a significant improvement in acquisition after 21 days of treatment (20 and 200 μg) over the vehicle control treated SAMP8 mice. While, as shown in Example 1, acute NNC 26-9100 (0.2 μg) improves learning and memory when given i.c.v., peripheral influences and chronic time-frame of delivery within this examination would account for the increase dose to induce a similar end-effect. With dosing continued up to 28 days, animals were then assessed for retention. Again, the 20 and 200 μg doses showed a significant increase in completion of the memory task. This shows NNC 26-9100 enhances learning and memory over a consistent i.p. administered dose.

Brain tissues of the NNC 26-9100 treated SAMP8 mice (post-memory testing) identified a decrease in A1342 at the 20 μg dose, which corresponded to an enhancement in learning and memory. However, we did not observe a similar decrease in A1342 at the 200 μg dose. The dose discrepancy between the learning-memory testing and A1342 levels may be indicative of dual SSTR4 activity. A dose associated impact on A1342 degradation could be independent of direct SSTR4 mechanisms governing learning and memory. A recent investigation in wild-type mice indicated hippocampal SSTR4 activity is selectively involved in memory and associated behavioral responses (Gastambide et al., 2009). Others have demonstrated somatostatin augments long-term potentiation, a primary process in learning and memory, via hippocampal mediated mechanisms (Matsuoka et al., 1991; Tallent, 2007). Thus, the dosing disparity observed for NNC 26-9100 may be the result of differential SSTR4 activity (i.e. Aβ degradation and/or hippocampus enhancing learning and memory). In this regard, the 20 μg dose may enhance Aβ degradation via downstream activation of neprilysin or other enzymatic mechanism (i.e. insulin-degrading enzyme) (Ciaccio et al., 2009), as well as direct receptor action. However, the 200 μg dose of NNC 26-9100 may fall into a range that counters the Aβ degrading process, but still has direct receptor activity with regards to learning and memory.

Brain tissues of the NNC 26-9100 treated SAMP8 mice (post-memory testing) identified no significant changes in protein expression for SSTR4, APP, or NEP across the dosing range. This is consistent with the single dose i.c.v. administration data as shown in Example 1. However, a critical observation is that over the 28 day period of treatment, no down-regulation of SSTR4 expression was observed, which would otherwise negatively impact long term use of such agonists. Additionally, no upregulation of APP expression was shown, which would also be beneficial in regards to long term treatment. Other laboratories have shown NEP degrades Aβ without affecting APP (Leissring et al., 2003; Madani et al., 2006; Poirier et al., 2006), which is consistent with the lack of change in APP expression we observed. While the NEP expression would be expected to be altered by the proposed feed-back mechanism of SSTR4 activation, NEP activity alterations may be different from the overall expression levels. Lastly, the observed impact of Aβ42 could be independent of NEP.

Thus these results identify the selective high-affinity SSTR4 agonist NNC 26-9100 crosses the BBB, with limited capillary sequestration, to primary areas within the brain associated with learning, memory and AD pathology. Furthermore, chronic i.p. administration induced a discernable enhancement of learning and memory in an established mouse model of cognitive decline associated with increased Aβ levels. While additional examinations are required to elucidate the full impact of NNC 26-9100 on enzymatic activation of neprilysin, Aβ42 was shown to decrease with the 20 μg dose of chronic NNC 26-9100. Taken together, these findings indicate that NNC 26-9100 is viable as a peripherally administered drug, capable of enhancing learning and memory. Other agonists directed at SSTR4 may also provide a viable therapy option in the treatment of Alzheimer's disease and/or other forms of cognitive impairment. The compound of Formula II (LIU 686-06A) was thus also investigated for effects in learning and memory.

LIU 686-06A Learning and Memory.

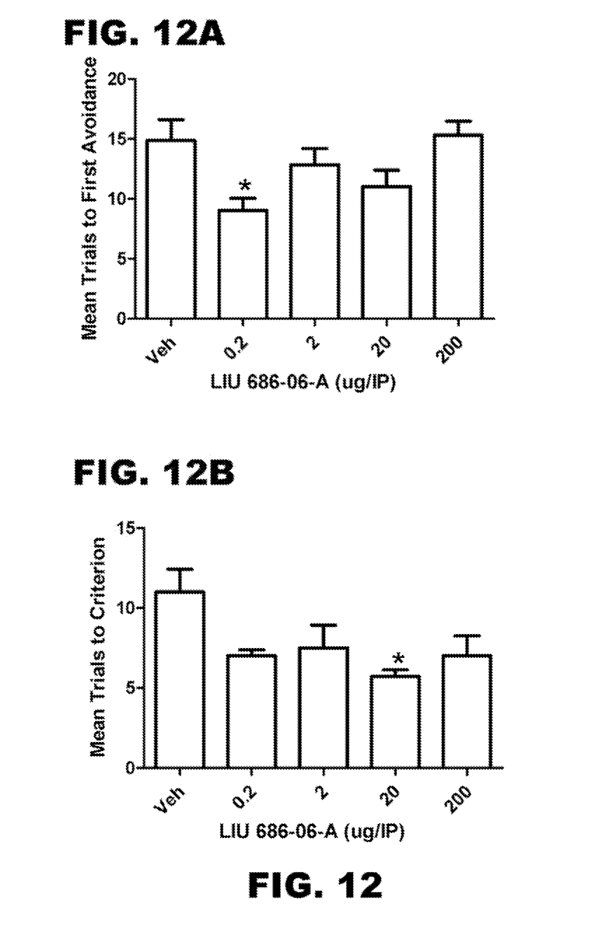

The t-maze as described above was also used to test for the effects of chronic peripheral LIU 686-06A administration on learned acquisition and retention in 12 month SAMP8 mice. On day 21 of treatment as described above, a decrease in the mean trials to first avoidance was observed, with the 0.2 μg daily dose showing a significantly lower number of mean trials to first avoidance (p<0.05) compared to vehicle control, demonstrating an improvement in learning (FIG. 12A). On day 21 of treatment, a decrease in the mean trials to criterion was observed, with the 20 μg daily dose showing a significantly lower number of mean trials to first criterion (p<0.05) compared to vehicle control, also demonstrating an improvement in learning (FIG. 12B). Thus LIU 686-06A also demonstrates effects on learned acquisition and retention, similar to those observed for NNC 26-9100.

### Example 3

### Effects of Acute NNC 26-9100 (i.c.v.) on Acquisition (Learning) and Recognition (Memory) in APPswe Transgenic Mouse

AAPswe Transgenic Mouse Model.

The effects of i.c.v. administration of NNC 26-9100 were also evaluated in AAPswe transgenic mice. The APPswe transgenic mouse carries a transgene coding for the 695-amino acid isoform of human Alzheimer A R precursor protein. (K. Hsiao et al., 274 Science 99-102, 1996). These mice express high concentrations of the mutant AR , with significant amyloid plaques, and display memory deficits. At 11-13 months of age they show a 14-fold increase in AR (1-42/43) over those at 2-8 months of age. Elevated AR levels are associated with the development of amyloid deposits in frontal, temporal and entorhinal cortex, hippocampus, presubiculum, and cerebellum, with some mice developing the signature cross pattern of deposits seen inhuman AD. Memory deficits have been demonstrated in 9-10 month old transgenic mice via altered performance in Y maze and Morris water mazes, correlating with the development of amyloid plaques. Thus APPswe transgenic mice are also a good model for studying APP expression, amyloid plaque formation, neuronal decline, and memory loss associated with AD, and for studying drug candidates for treatment or prevention of AD. APPswe (Tg2576) mice, and non-transgenic siblings as controls, were obtained from Taconic (New York). All other materials and methods were as described for Example 1.

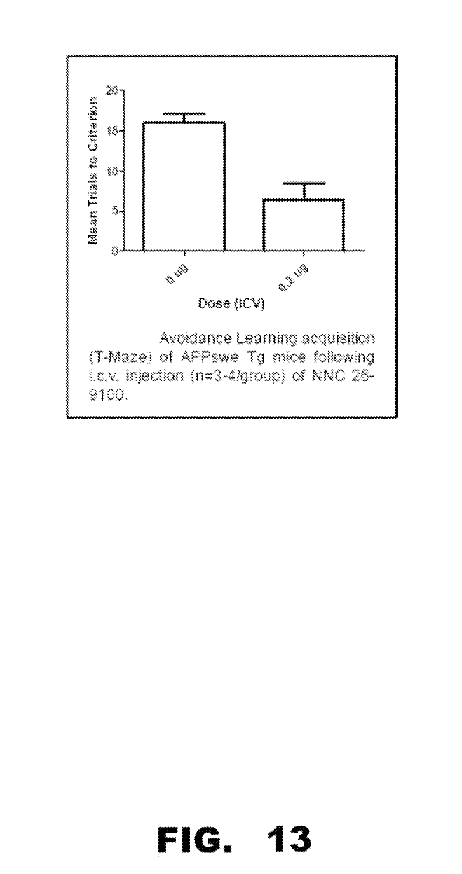

AAPswe transgenic mice were administered 0.2 μg of NNC 26-91001.c.v. after a 2 hour fasting period, followed by an additional hour of fasting after administration. Animals were tested using the T-maze model as described above in Example 1. FIG. 13 is a bar graph of results for avoidance learning acquisition in controls (0 μg) and treated (0.2 μg) AAPswe transgenic mice, showing that NNC 26-9100 when acutely administered had effects in AAPswe transgenic mice similar to those obtained in SAMP8 mice.

### Example 4

### Synthesis of 3-(3-1H-Imidazol-4-yl)propyl-1-(2-(1H-indol-3-yl)ethyl-1-(3,4-dichlorobenzyl)urea (Formula III)

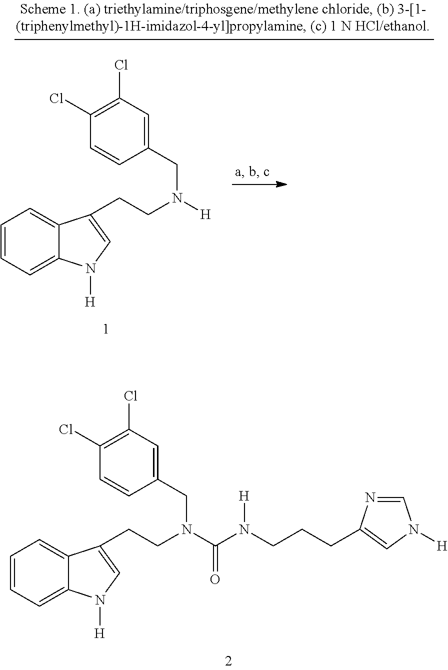

Synthesis of 3-(3-1H-Imidazol-4-yl)propyl-1-(2-(1H-indol-3-yl)ethyl-1-(3,4-dichlorobenzyl)urea (2) was performed as follows. N-(3,4-Dichlorobenzyl)-2-(1H-indol-3-yl)ethanamine (1, 696 mg, 2.18 mmol) was suspended in methylene chloride (20 mL) and triethylamine (560 mg, 0.78 mL, 5.54 mmol) was added under nitrogen. The stirred solution was treated with triphosgene (259 mg, 0.87 mmol) in methylene chloride (20 mL) over 30 minutes. After 30 minutes, additional triethylamine (800 mg, 2.18 mmol) was added, and the reaction mixture was stirred overnight. The volatiles were removed under reduced pressure, and the residue was dissolved in ethyl acetate (100 mL). The ethyl acetate was washed with water (3×50 mL), dried (sodium sulfate), filtered, and evaporated to yield a light brown residue. Purification by flash chromatography (silica gel) using methylene chloride-methanol (98:2) gave 1.47 g of a light yellow oil. The oil was suspended in 1N hydrochloric acid (35 mL), and ethanol was added to obtain a clear solution. After refluxing for 7 hours, the reaction mixture was cooled to room temperature and stirred overnight under nitrogen. The reaction mixture was filtered, and the ethanol was removed under reduced pressure. The resulting acidic solution was basified with 1N sodium hydroxide and extracted with methylene chloride (3×50 mL), dried (sodium sulfate), and evaporated under reduced pressure to yield a colorless oil. The oil was placed under high vacuum for several hours to yield 167 mg (16%) of 2 as a light yellow foam. TLC (silica gel, methylene chloride 95: methanol 5: ammonium hydroxide 1) indicated an essentially pure compound.: ¹H NMR δ 1.34 (1.34 m, 2H), 2.23 (t, J=5.95 Hz, 2H), 3.51 (t, J=5.95 Hz, 2H), 4.31 (t, J=5.50 Hz, 1H), 4.46 (s, 2H), 6.65 (s, 1H), 6.97 (br s, 1H), 7.10 (m, 3H), 7.34 (m, 3H), 7.47 (s, 1H), 7.56 (d, J=7.79 Hz, 1H), 9.23 (s, 1H); ¹³C NMR δ 21.77, 24.18, 30.52, 39.07, 47.67, 49.12, 111.78, 112.13, 117.99, 122.52, 123.17, 126.73, 126.93, 127.99, 128.06, 129.43, 130.65, 131.15, 132.74, 134.50, 136.74, 138.90, 159.33.

### REFERENCES

1. Ankersen M, Crider A M, Liu S, Ho B, Andersen H S and Stidsen C E (1998) Discovery of a Novel Non-Peptide Somatostatin Agonist with SST4 Selectivity. Journal of the American Chemical Society 120:1368-1373.
2. Banks W A (2009) Characteristics of compounds that cross the blood-brain barrier. BMC Neurol 9 Suppl 1:S3.
3. Banks W A, Robinson S M, Diaz-Espinoza R, Urayama A and Soto C (2009) Transport of prion protein across the blood-brain barrier. Exp Neurol 218:162-167.
4. Bevins R A and Besheer J (2006) Object recognition in rats and mice: a one-trial nonmatching- to-sample learning task to study ‘recognition memory’. Nat Protoc 1:1306-1311.
5. Bruno J F, Xu Y, Song J and Berelowitz M (1992) Molecular cloning and functional expression of a brain-specific somatostatin receptor. Proc Natl Acad Sci USA 89:11151-11155.
6. Cervia D and Bagnoli P (2007) An update on somatostatin receptor signaling in native systems and new insights on their patho physiology. Pharmacol Ther 116:322-341.
7. Ciaccio C, Tundo G R, Grasso G, Spoto G, Marasco D, Ruvo M, Gioia M, Rizzarelli E and Coletta M (2009) Somatostatin: a novel substrate and a modulator of insulin-degrading enzyme activity. J Mol Biol 385:1556-1567.
8. Crider A M, Liu S, Li T, Mahajan S, Ankersen M and Stidsen C E (2004) Somatostatin receptor subtype 4 (sst4) ligands: Synthesis and evaluation of indol-3-yl- and 2-pyridylthioureas. Letters in Drug Design & Discovery 1:84-87.
9. Davies P, Katzman R and Terry R D (1980) Reduced somatostatin-like immunoreactivity in cerebral cortex from cases of Alzheimer disease and Alzheimer senile dementia. Nature 288:279-280.
10. Epelbaum J, Guillou J L, Gastambide F, Hoyer D, Duron E and Viollet C (2009) Somatostatin, Alzheimer's disease and cognition: An old story coming of age? Prog Neurobiol.
11. Farr S A, Banks W A, La Scola M E, Flood J F and Morley J E (2000) Permanent and temporary inactivation of the hippocampus impairs T-maze footshock avoidance acquisition and retention. Brain Res 872:242-249.
12. Flood J F and Morley J E (1993) Age-related changes in footshock avoidance acquisition and retention in senescence accelerated mouse (SAM). Neurobiol Aging 14:153-157.
13. Fontan-Lozano A, Romero-Granados R, Troncoso J, Munera A, Delgado-Garcia J M and Carrion A M (2008) Histone deacetylase inhibitors improve learning consolidation in young and in KA-i nduced-neu rodege ne ration and SAMP-8-mutant mice. Mol Cell Neurosci 39:193-201.
14. Gastambide F, Viollet C, Lepousez G, Epelbaum J and Guillou J L (2009) Hippocampal SSTR4 somatostatin receptors control the selection of memory strategies. Psychopharmacology (Berl) 202:153-163.
15. Hama E and Saido T C (2005) Etiology of sporadic Alzheimer's disease: somatostatin, neprilysin, and amyloid beta peptide. Med Hypotheses 65:498-500.
16. Hayashi M, Yamashita A and Shimizu K (1997) Somatostatin and brain-derived neurotrophic factor mRNA expression in the primate brain: decreased levels of mRNAs during aging. Brain Res 749:283-289.
17. Iwata N, Mizukami H, Shirotani K, Takaki Y, Muramatsu S, Lu B, Gerard N P, Gerard C, Ozawa K and Saido T C (2004) Presynaptic localization of neprilysin contributes to efficient clearance of amyloid-beta peptide in mouse brain. J Neurosci 24:991-998.
18. Iwata N, Takaki Y, Fukami S, Tsubuki S and Saido T C (2002) Region-specific reduction of A beta-degrading endopeptidase, neprilysin, in mouse hippocampus upon aging. J Neurosci Res 70:493-500.
19. Iwata N, Higuchi M and Saido T C (2005) Metabolism of amyloid-beta peptide and Alzheimer's disease. Pharmacol Ther 108:129-148.
20. Iwata N, Tsubuki S, Takaki Y, Shirotani K, Lu B, Gerard N P, Gerard C, Hama E, Lee H J and Saido T C (2001) Metabolic regulation of brain Abeta by neprilysin. Science 292:1550-1552.
21. Iwata N, Tsubuki S, Takaki Y, Watanabe K, Sekiguchi M, Hosoki E, Kawashima-Morishima M, Lee H J, Hama E, Sekine-Aizawa Y and Saido T C (2000) Identification of the major Abeta1-42-degrading catabolic pathway in brain parenchyma: suppression leads to biochemical and pathological deposition. Nat Med 6:143-150.
22. Kumar U (2005) Expression of somatostatin receptor subtypes (SSTR1-5) in Alzheimer's disease brain: an immunohistochemical analysis. Neuroscience 134:525-538.
23. Kumar V B, Farr S A, Flood J F, Kamlesh V, Franko M, Banks W A and Morley J E (2000) Site-directed antisense oligonucleotide decreases the expression of amyloid precursor protein and reverses deficits in learning and memory in aged SAMP8 mice. Peptides 21:1769-1775.
24. Lanz T A and Schachter J B (2006) Demonstration of a common artifact in immunosorbent assays of brain extracts: development of a solid-phase extraction protocol to enable measurement of amyloid-beta from wild-type rodent brain. J Neurosci Methods 157:71-81.
25. Leissring M A, Farris W, Chang A Y, Walsh D M, Wu X, Sun X, Frosch M P and Selkoe D J (2003) Enhanced proteolysis of beta-amyloid in APP transgenic mice prevents plaque formation, secondary pathology, and premature death. Neuron 40:1087-1093.
26. Madani R, Poirier R, Wolfer D P, Welzl H, Groscurth P, Lipp H P, Lu B, El Mouedden M, Mercken M, Nitsch R M and Mohajeri M H (2006) Lack of neprilysin suffices to generate murine amyloid-like deposits in the brain and behavioral deficit in vivo. J Neurosci Res 84:1871-1878.
27. Mathuranath, P. S. et al., Neurology. 2000 Dec. 12; 55(11):1613-20.
28. Matsuoka N, Kaneko S and Satoh M (1991) Somatostatin augments long-term potentiation of the mossy fiber-CA3 system in guinea-pig hippocampal slices. Brain Res 553:188-194.
29. Mohajeri M H and Wolfer D P (2009) Neprilysin deficiency-dependent impairment of cognitive functions in a mouse model of amyloidosis. Neurochem Res 34:717-726.
30. Moller L N, Stidsen C E, Hartmann B and Hoist J J (2003) Somatostatin receptors. Biochim Biophys Acta 1616:1-84.
31. Morley J E, Farr S A and Flood J F (2002) Antibody to amyloid beta protein alleviates impaired acquisition, retention, and memory processing in SAMP8 mice. Neurobiol Learn Mem 78:125-138.
32. Morley J E, Kumar V B, Bernardo A E, Farr S A, Uezu K, Tumosa N and Flood J F (2000) Beta-amyloid precursor polypeptide in SAMP8 mice affects learning and memory. Peptides 21:1761-1767.
33. Mouri A, Zou L B, Iwata N, Saido T C, Wang D, Wang M W, Noda Y and Nabeshima T (2006) Inhibition of neprilysin by thiorphan (i.c.v.) causes an accumulation of amyloid beta and impairment of learning and memory. Behav Brain Res 168:83-91.
34. Nisemblat Y, Belinson H, Dolev I and Michaelson D M (2008) Activation of the amyloid cascade by intracerebroventricular injection of the protease inhibitor phosphoramidon. Neurodegener Dis 5:166-169.
35. Oldendorf W H (1974) Lipid solubility and drug penetration of the blood brain barrier. Proc Soc Exp Biol Med 147:813-815.
36. Patlak C S, Blasberg R G and Fenstermacher J D (1983) Graphical evaluation of blood-tobrain transfer constants from multiple-time uptake data. J Cereb Blood Flow Metab 3:1-7.
37. Poirier R, Wolfer D P, Welzl H, Tracy J, Galsworthy M J, Nitsch R M and Mohajeri M H (2006) Neuronal neprilysin overexpression is associated with attenuation of Abeta-related spatial memory deficit. Neurobiol Dis.
38. Poon H F, Joshi G, Sultana R, Farr S A, Banks W A, Morley J E, Calabrese V and Butterfield D A (2004) Antisense directed at the Abeta region of APP decreases brain oxidative markers in aged senescence accelerated mice. Brain Res 1018:86-96.
39. Saito T, Iwata N, Tsubuki S, Takaki Y, Takano J, Huang S M, Suemoto T, Higuchi M and Saido T C (2005) Somatostatin regulates brain amyloid beta peptide Abeta42 through modulation of proteolytic degradation. Nat Med 11:434-439.
40. Sandoval K E, Farr S A, Banks W A, Crider A M and Witt K ((Co-submitted manuscript)) Intracerebrovascular Administration of Somatostatin Receptor Subtype-4 Agonist NNC 26-9100 Enhances Learning and Memory with Altered Amyloid Beta.
41. Sano M, Grossman H and Van Dyk K (2008) Preventing Alzheimer's disease: separating fact from fiction. CNS Drugs 22:887-902.
42. Selkoe D J (2008) Soluble oligomers of the amyloid beta-protein impair synaptic plasticity and behavior. Behav Brain Res 192:106-113.
43. Shirotani K, Tsubuki S, Iwata N, Takaki Y, Harigaya W, Maruyama K, Kiryu-Seo S, Kiyama H, Iwata H, Tomita T, Iwatsubo T and Saido T C (2001) Neprilysin degrades both amyloid beta peptides 1-40 and 1-42 most rapidly and efficiently among thiorphan- and phosphoramidon-sensitive endopeptidases. J Biol Chem 276:21895-21901.
44. Strittmatter M, Cramer H, Reuner C, Strubel D, Hamann G and Schimrigk K (1997) Molecular forms of somatostatin-like immunoreactivity in the cerebrospinal fluid of patients with senile dementia of the Alzheimer type. Biol Psychiatry 41:1124-1130.
45. Tallent M K (2007) Somatostatin in the dentate gyrus. Prog Brain Res 163:265-284.
46. Tomobe K and Nomura Y (2009) Neurochemistry, neuropathology, and heredity in SAMP8: a mouse model of senescence. Neurochem Res 34:660-669.
47. Triguero D, Buciak J and Pardridge W M (1990) Capillary depletion method for quantification of blood-brain barrier transport of circulating peptides and plasma proteins. J Neurochem 54:1882-1888.
48. Yasojima K, Akiyama H, McGeer E G and McGeer P L (2001a) Reduced neprilysin in high plaque areas of Alzheimer brain: a possible relationship to deficient degradation of betaamyloid peptide. Neurosci Lett 297:97-100.
49. Yasojima K, McGeer E G and McGeer P L (2001b) Relationship between beta amyloid peptide generating molecules and neprilysin in Alzheimer disease and normal brain. Brain Res 919:115-121.
50. Ziegler-Graham K, Brookmeyer R, Johnson E and Arrighi H M (2008) Worldwide variation in the doubling time of Alzheimer's disease incidence rates. Alzheimers Dement 4:316-323.
51. Zupa-Fernandez A, Rozzi A M, Arnold H M, Felsenstein K M, Rowley A, Treton G and Yohrling G (2007) Optimization of soluble amyloid beta extraction from non-transgenic rodents to facilitate the testing of gamma-secretase modulators, in Society for Neuroscience, 37th, San Diego.

What is claimed is:

1. A method for treating memory impairment from Alzheimer's disease in a subject in need thereof, comprising administering to the subject a therapeutically effective amount of a somatostatin receptor-4 (SSTR4) agonist.

2. The method of claim 1 wherein the SSTR4 agonist is a compound of formula I:

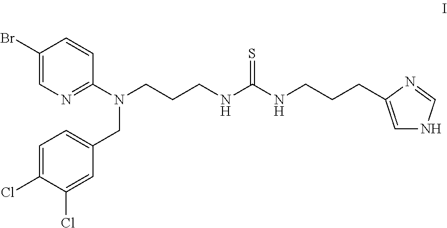

3. The method of claim 1 wherein the SSTR4 agonist is a compound of formula II:

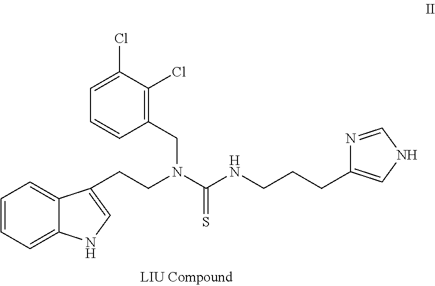

4. The method of claim 1 wherein the SSTR4 agonist is a compound of formula III:

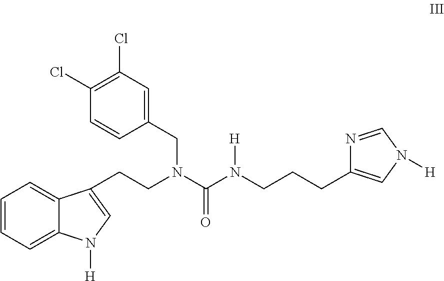

5. The method of claim 1 wherein the therapeutically effective amount is from 0.01 mg/kg to about 10 mg/kg of body weight per day.

6. The method of claim 1 wherein the somatostatin receptor-4 (SSTR4) agonist is administered to the subject through a peripheral administration route.

7. The method of claim 6 wherein the peripheral route of administration is selected from the group consisting of intraperitoneal, intravenous, intramuscular and intraarterial.

8. The method of claim 1 wherein the somatostatin receptor-4 (SSTR4) agonist is administered in an intravenous dosage form further comprising a pharmaceutically acceptable carrier.

9. The method of claim 1 wherein the somatostatin receptor-4 (SSTR4) agonist is administered in an intraperitoneal dosage form further comprising a pharmaceutically acceptable carrier.

10. A method of improving memory function in a subject suffering from Alzheimer's disease, the method comprising:
a. determining a first level of memory function in the subject;
b. administering to the subject a therapeutically effective amount of a somatostatin receptor-4 (SSTR4) agonist; and
c. determining a second level of memory function following administration of the SSTR4 agonist;
d. comparing the second level of memory function in the subject to the first level of memory function in the subject such that a detectable improvement in memory function indicates that the subject is responding positively to the administration of the SSTR4 agonist.

11. The method of claim 10 wherein the SSTR4 agonist is a compound of formula I:

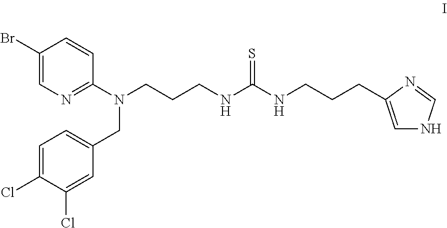

12. The method of claim 10 wherein the SSTR4 agonist is a compound of formula II:

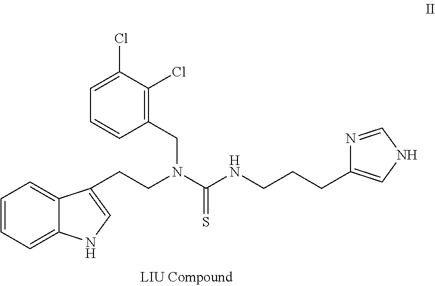

13. The method of claim 10 wherein the SSTR4 agonist is a compound of formula III:

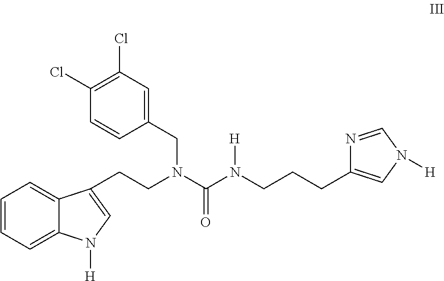

14. The method of claim 10 wherein the therapeutically effective amount is from 0.01 mg/kg to about 10 mg/kg of body weight per day.

15. The method of claim 10 wherein the somatostatin receptor-4 (SSTR4) agonist is administered to the subject through a peripheral administration route.

16. The method of claim 15 wherein the peripheral route of administration is selected from the group consisting of intraperitoneal, intravenous, intramuscular and intraarterial.

17. The method of claim 10 wherein the somatostatin receptor-4 (SSTR4) agonist is administered in an intravenous dosage form further comprising a pharmaceutically acceptable carrier.

18. The method of claim 10 wherein the somatostatin receptor-4 (SSTR4) agonist is administered in an intraperitoneal dosage form further comprising a pharmaceutically acceptable carrier.

19. A method to improve memory function in a subject suffering from Alzheimer's disease, the method comprising reducing A_{R42} levels in brain tissue of the subject by administering to the subject a therapeutically effective amount of a somatostatin receptor-4 (SSTR4) agonist.

20. The method of claim 19 wherein the SSTR4 agonist is a compound of formula I:

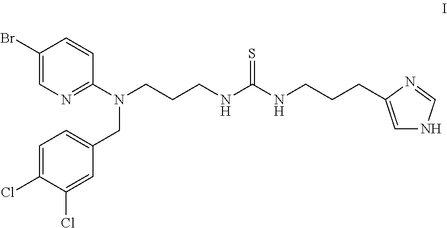

21. The method of claim 19 wherein the SSTR4 agonist is a compound of formula II:

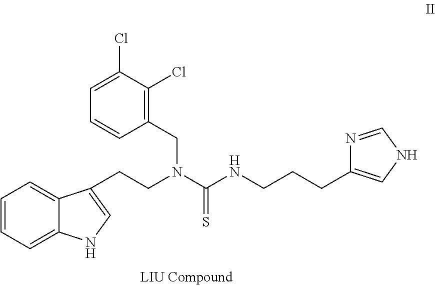

22. The method of claim 19 wherein the SSTR4 agonist is a compound of formula III:

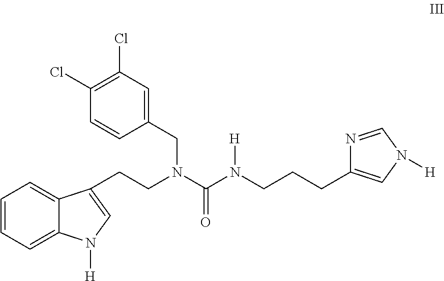

23. The method of claim 19 wherein the therapeutically effective amount is from 0.01 mg/kg to about 10 mg/kg of body weight per day.

24. The method of claim 19 wherein the somatostatin receptor-4 (SSTR4) agonist is administered to the subject through a peripheral administration route.

25. The method of claim 24 wherein the peripheral route of administration is selected from the group consisting of intraperitoneal, intravenous, intramuscular and intraarterial.

26. The method of claim 19 wherein the somatostatin receptor-4 (SSTR4) agonist is administered in an intravenous dosage form further comprising a pharmaceutically acceptable carrier.

27. The method of claim 19 wherein the somatostatin receptor-4 (SSTR4) agonist is administered in an intraperitoneal dosage form further comprising a pharmaceutically acceptable carrier.

28. A compound of formula III:

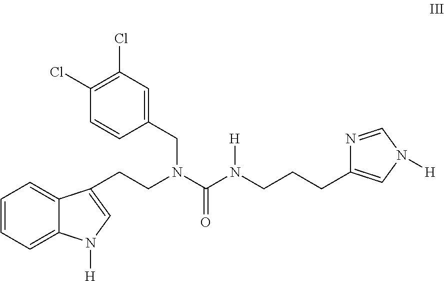

29. A pharmaceutical composition comprising the compound of claim 28 and a pharmaceutically acceptable carrier, excipient or diluent.
